(19) United States
(12) Patent Application Publication
(10) Pub. No.: US 2009/0074685 A1
(43) Pub. Date: Mar. 19, 2009

(54) POLYAMIDE AND EMOLLIENT COMPOSITIONS, PRODUCTS MADE THEREFROM, AND METHODS OF MAKING AND USING SUCH COMPOSITIONS AND PRODUCTS

(76) Inventor: Kuo-Tsai Griffin Lai, Rincon, GA (US)

Correspondence Address: ARIZONA CHEMICAL COMPANY;c/o Bo Segers, P.O. Box 550850, Jacksonville, FL, 32255, US

(21) Appl. No.: 12/212,071

(22) Filed: Sep. 17, 2008

Related U.S. Application Data
(60) Provisional application No. 60/973,155, filed on Sep. 17, 2007.
(60) Provisional application No. 60/988,769, filed on Nov. 16, 2007.
(60) Provisional application No. 60/990,275, filed on Nov. 26, 2007.

(51) Int. Cl. A61K 8/88; A61K 47/32; A61Q 13/00; A61Q 17/04; A61Q 1/06; A61Q 15/00; A61Q 19/04; A61Q 19/10; A61Q 17/02

(52) U.S. Cl. 424/59; 514/772.3; 424/63; 424/64; 424/65; 510/445; 512/4

(57) ABSTRACT

Formulations comprising a polyamide and an emollient, wherein the emollient comprises at least one selected from the group consisting of hydrocarbons with more than 8 carbons that may be linear or branched, and saturated or unsaturated; esters, such as benzoate esters, simple esters, or bulky esters; glycerol or glycerol condensate (mono-, di- or tri-); polyglycerol or polyglycerol condensate; guerbet ester; vegetable oils; and mixtures of silicone oils with any of the above emollients. Formulations may be any of cosmetic formulations, personal care product formulations, and all combinations thereof. Products comprising said formulations, and methods of making and using said formulations and products.

### REFERENCE TO RELATED APPLICATIONS

The present application claims priority of U.S. Provisional Patent Application Ser. No. 60/973,155, filed Sep. 17, 2007, and U.S. Provisional Patent Application Ser. No. 60/988,769, filed Nov. 16, 2007, and U.S. Provisional Patent Application Ser. No. 60/990,275, filed Nov. 26, 2007, all of which are incorporated herein by reference.

### BACKGROUND OF THE INVENTION

1. Field of the Invention

The present invention relates to polyamide and emollient compositions, to products made therefrom, and to methods of making and using such compositions and products. In another aspect, the present invention relates to polyamide and emollient compositions useful in personal care products such as cosmetics, skin care and pharmaceuticals, to personal care products made therefrom, and methods of making and using such compositions and products. In even another aspect, the present invention relates to polyamide and emollient compositions useful as precursors in making personal care products such as cosmetics, skin care and pharmaceuticals, to personal care products made therefrom, and methods of making and using such compositions and products.

2. Brief Description of the Related Art

In many commercially important compositions, the consistency of the product is critical to its commercial success. One example is personal care products, which generally contain one or more active ingredients within a carrier formulation. While the active ingredient(s) determine the ultimate performance properties of the product, the carrier formulation is equally critical to the commercial success of the product in that it largely determines the consistency of the product. The rheology of the carrier (also referred to as the “base”) largely determines the flow properties of the product, and the flow properties largely determine the manner in which the consumer will apply or use the product.

For example, aluminum chlorohydrate, aluminum-zirconium tetrachlorohydrate, aluminum-zirconium polychlorohydrate complexed with glycine, and aluminum-zirconium complexed with any of trichlorohydrate, octachlorohydrate, and sesquichlorohydrate are metal salts that are commonly used as active ingredients in deodorant and antiperspirant products. Consumers have generally shown a preference for applying deodorant from a stick form. Thus, the carrier in a stick-form deodorant must be a relatively hard substance, and waxy fatty alcohol such as stearyl alcohol has often been used as the carrier in these products. As another example, the active ingredient in a lipstick is the colorant. A lipstick should not be as hard as a stick deodorant, but of course must maintain its shape when undisturbed at room temperature. A blend of wax and oil is known to provide a consistency that is well-suited as a carrier for a lipstick. As a final example, shampoo desirably has a viscosity greater than water, and when the active ingredient(s) in a shampoo does not have a sufficiently high viscosity, a somewhat viscous carrier material is desirably included in the shampoo formulation.

From the above examples, it can be seen that formulators of personal care products depend upon the availability of materials having various rheological properties in order to formulate a successful personal care product. Materials that have a gel-like character, in that they maintain their shape when undisturbed but flow upon being rubbed, are often desired for personal care products.

Transparent (i.e., clear) bases or carriers are desired by formulators who develop a personal care product wherein colorant is an active ingredient, because a transparent carrier (as opposed to an opaque carrier) will minimally, if at all, interfere with the appearance of the colorant. In recent years, consumers have demonstrated an increasing preference for transparent and colorless personal care products such as deodorants and shampoos. There is thus an increasing demand for transparent materials that can provide the rheological properties needed for various personal care products, and particularly which can impart gel-like character to a formulation.

Polyamide resin prepared from polymerized fatty acid and diamine is reported to function as a gellant in formulations developed for personal care products. For example, U.S. Pat. No. 3,148,125 is directed to a clear lipstick carrier composition formed from polyamide resin compounded with a lower aliphatic alcohol and a so-called “polyamide solvent.” Likewise, U.S. Pat. No. 5,500,209 is directed to forming a gel or stick deodorant, where the composition contains polyamide gelling agent and a solvent system including monohydric or polyhydric alcohols.

Polyamide resins are typically synthesized by reacting dicarboxylic acid-containing compounds with diamine-containing compounds. This reaction produces repeating hydrocarbon subunits connected to each other through amide bond linkages. Nylon is one type of particularly well-known polyamide, although there are many other types of polyamide compounds possessing various strength, flexibility, and solubility properties, due largely to the lengths of the hydrocarbon portions of the reacting species.

One particularly useful type of polyamide is made from polymerized fatty acid, also referred to as dimer acid. These polyamides may be referred to as polymerized fatty acid-based polyamides, or dimer acid-based polyamides. Sometimes these polyamides are also referred to polymerized fatty acid-containing polyamides, however, it should be noted that polymerized fatty acid per se is not present to any great extent in these polyamides, and that the term “polymerized fatty acid-containing” is really a shorthand expression that denotes that the polyamides were prepared from polymerized fatty acid. Regardless of their name, in structural terms these polyamides contain longer hydrocarbon regions than are found in nylons, and may be terminated at each end by organic functional moieties such as amide and ester groups. Generally, the molecular structures of polymerized fatty acid-based polyamides impart to these resins properties of strength and flexibility, making them particularly useful in formulations for protective coatings and gelling agents. Indeed, articles of commerce such as candles, air fresheners, and cable protectants can be manufactured with the use of such resins.

Because of the prominence of the hydrocarbon regions in typical polymerized fatty acid-based polyamides, such resins are often combined with low-polarity components, such as mineral oils and cosmetic-grade esters.

Despite advances in the art, there remains a need for formulations and compositions suitable for use in the personal care product industry.

### SUMMARY OF THE INVENTION

The following presents a general summary of some of the many possible embodiments of this disclosure in order to provide a basic understanding of this disclosure. This summary is not an extensive overview of all embodiments of this disclosure. This summary is not intended to identify key or critical elements of the disclosure or to delineate or otherwise limit the scope of the claims. The following summary merely presents some concepts of the disclosure in a general form as a prelude to the more detailed description that follows.

According to one embodiment of the present invention, there is provided a cosmetic formulation comprising a polyamide and an emollient. The emollient comprises at least one selected from the group consisting of hydrocarbons with more than 8 carbons that may be linear or branched, and saturated or unsaturated; esters, such as benzoate esters, simple esters, or bulky esters; glycerol or glycerol condensate (mono-, di- or tri-); polyglycerol or polyglycerol condensate; guerbet ester; vegetable oils; and mixtures of silicone oils with any of the above emollients. In further embodiments of this embodiment, the emollient comprises at least one selected from the group consisting of isooctane; polyisobutene and isododecane; isododecane; isohexadecane; isoeicosane; polyisobutene; hydrogenated polyisobutene; hydrogenated polyisobutene; paraffin; isoparaffins; petrolatum; limonene; squalene; C12-C15 alkyl benzoate; isostearyl benzoate; octyl benzoate; dipropylene glycol benzoate; C12-15 alkyl benzoate and dipropylene glycol dibenzoate and stearyl ether benzoate; glycereth-7 benzoate; octyldodecyl neopentanoate; PEG-4 diheptanoate; C12-15 alkyl lactate; benzyl laurate; octyl isononanoate; ethylhexyl isononanoate; bis trioctyldodecyl citryl; caprylic/capric triglyceride; glycereth-7 triacetate; triisostearin; isostearyl palmitate; isocetyl stearate; isononyl isononanoate; neopentyl glycol dicaprylate/dicaprate; octyldodecyl ricinoleate; ethylhexyl palmitate; octyl stearate; trimethylpropane triethylhexanoate; dioctyl sebacate; isopropyl palmitate; isopropyl myristate; isodecyl neopentanoate; isodecyl oelate; neopentyl glycol distearate; 2-ethylhexyl salicyclate; caprylic/capric triglyceride; PEG-1 glycerol sorbitan isostearate; dioctyl malate; dioctyl/capryl adipate; diisopropyl sebacate; dioleyl malate; diisostearyl fumarate; diisopropyl dimer dilinoleate; dimer dilinoleyl dimer dilinoleate; dipentaerythrityl tetrahydroxystearate/isostearate; dipentaerythrityl tetrabehenate polyhydroxystearate; triisostearyl citrate; triisostearyl trilinoleate; dioctadecanyl didecyletetradecanoate; dioctadecanyl ditetradecyloctadecanoate; neopentyl glycol diethylhexanoate; neopentyl glycol dicaprate; triisostearyl citrate; stearyl erucate; behenyl erucate; isosstearyl hydroxystearate; ethylhexyl hydroxystearate; cetearyl ethylhexanoate; isostearyl isostearate; PEG-90 diisostearate; PEG/PPG-8/3 diisostearate; PEG/PPG-8/3 laurate; glycerol behenate; glycerol eicosanate; glycerol behenate eicosanate; glyceryl behenate/eicosadioate; glycerol isostearate; glyceryl tri(2-ethylhexanoate); polyglyceryl-10 behenate/eicosadioate; polyglyceryl-6 behenate; polyglyceryl-6 octastearate; polyglyceryl-6 polyricinoleate; polygyceryl-3 laurate; diisostearoyl polyglyceryl-3 dimer dilinoleate; triisostearoyl polyglyceryl-3 dimer dilinoleate; octyldodecyl ricinoleate; trioctyldodecyl citrate; castor oil; jojoba oil; corn oil; avocado oil; sunflower oil; soybean oil; olive oil; cottonseed oil; grape seed oil; palm oil; sesame seed oil; almond oil; methyl soyate; isocetyl alcohol; dipropylene glycol monomethyl ether; dipropylene glycol dimethyl ether; and dipropylene glycol methyl acetate. in one non-limiting embodiment, the emollient comprises at least glyceryl behenate/eicosadioate or polyglyceryl-10 behenate/eicosadioate.

According to another embodiment of the present invention, there is provided a stick applied personal care product formulation comprising a colorant, a polyamide and an emollient. The emollient comprises at least one selected from the group consisting of hydrocarbons with more than 8 carbons that may be linear or branched, and saturated or unsaturated; esters, such as benzoate esters, simple esters, or bulky esters; glycerol or glycerol condensate (mono-, di- or tri-); polyglycerol or polyglycerol condensate; guerbet ester; vegetable oils; and mixtures of silicone oils with any of the above emollients. In further embodiments of this embodiment, the emollient comprises at least one selected from the group consisting of isooctane; polyisobutene and isododecane; isododecane; isohexadecane; isoeicosane; polyisobutene; hydrogenated polyisobutene; hydrogenated polyisobutene; paraffin; isoparaffins; petrolatum; limonene; squalene; C12-C15 alkyl benzoate; isostearyl benzoate; octyl benzoate; dipropylene glycol benzoate; C12-15 alkyl benzoate and dipropylene glycol dibenzoate and stearyl ether benzoate; glycereth-7 benzoate; octyldodecyl neopentanoate; PEG-4 diheptanoate; C12-15 alkyl lactate; benzyl laurate; octyl isononanoate; ethylhexyl isononanoate; bis trioctyldodecyl citryl; caprylic/capric triglyceride; glycereth-7 triacetate; triisostearin; isostearyl palmitate; isocetyl stearate; isononyl isononanoate; neopentyl glycol dicaprylate/dicaprate; octyldodecyl ricinoleate; ethylhexyl palmitate; octyl stearate; trimethylpropane triethylhexanoate; dioctyl sebacate; isopropyl palmitate; isopropyl myristate; isodecyl neopentanoate; isodecyl oelate; neopentyl glycol distearate; 2-ethylhexyl salicyclate; caprylic/capric triglyceride; PEG-1 glycerol sorbitan isostearate; dioctyl malate; dioctyl/capryl adipate; diisopropyl sebacate; dioleyl malate; diisostearyl fumarate; diisopropyl dimer dilinoleate; dimer dilinoleyl dimer dilinoleate; dipentaerythrityl tetrahydroxystearate/isostearate; dipentaerythrityl tetrabehenate polyhydroxystearate; triisostearyl citrate; triisostearyl trilinoleate; dioctadecanyl didecyletetradecanoate; dioctadecanyl ditetradecyloctadecanoate; neopentyl glycol diethylhexanoate; neopentyl glycol dicaprate; triisostearyl citrate; stearyl erucate; behenyl erucate; isosstearyl hydroxystearate; ethylhexyl hydroxystearate; cetearyl ethylhexanoate; isostearyl isostearate; PEG-90 diisostearate; PEG/PPG-8/3 diisostearate; PEG/PPG-8/3 laurate; glycerol behenate; glycerol eicosanate; glycerol behenate eicosanate; glyceryl behenate/eicosadioate; glycerol isostearate; glyceryl tri(2-ethylhexanoate); polyglyceryl-10 behenate/eicosadioate; polyglyceryl-6 behenate; polyglyceryl-6 octastearate; polyglyceryl-6 polyricinoleate; polygyceryl-3 laurate; diisostearoyl polyglyceryl-3 dimer dilinoleate; triisostearoyl polyglyceryl-3 dimer dilinoleate; octyldodecyl ricinoleate; trioctyldodecyl citrate; castor oil; jojoba oil; corn oil; avocado oil; sunflower oil; soybean oil; olive oil; cottonseed oil; grape seed oil; palm oil; sesame seed oil; almond oil; methyl soyate; isocetyl alcohol; dipropylene glycol monomethyl ether; dipropylene glycol dimethyl ether; and dipropylene glycol methyl acetate. The stick applied personal care product may be any one or more selected from the group consisting of lip gloss, lip stick, lip moisturizer, stick deodorant, antiperspirant, stick sun block, stick tanning lotion, stick applied pharmaceuticals, stick soaps, stick perfumes, stick applied insect repellents. In one non-limiting embodiment, the emollient comprises at least glyceryl behenate/eicosadioate or polyglyceryl-10 behenate/eicosadioate. In another non-limiting embodiment, the emollient comprises at least glyceryl behenate/eicosadioate or polyglyceryl-10 behenate/eicosadioate, and the stick applied personal care product is selected from the group consisting of lip gloss, lip stick, and lip moisturizer.

According to another embodiment of the present invention, there is provided a cosmetic product comprising a cosmetically active ingredient, a polyamide and an emollient. The emollient comprises at least one selected from the group consisting of hydrocarbons with more than 8 carbons that may be linear or branched, and saturated or unsaturated; esters, such as benzoate esters, simple esters, or bulky esters; glycerol or glycerol condensate (mono-, di- or tri-); polyglycerol or polyglycerol condensate; guerbet ester; vegetable oils; and mixtures of silicone oils with any of the above emollients. In further embodiments of this embodiment, the emollient comprises at least one selected from the group consisting of isooctane; polyisobutene and isododecane; isododecane; isohexadecane; isoeicosane; polyisobutene; hydrogenated polyisobutene; hydrogenated polyisobutene; paraffin; isoparaffins; petrolatum; limonene; squalene; C12-C15 alkyl benzoate; isostearyl benzoate; octyl benzoate; dipropylene glycol benzoate; C12-15 alkyl benzoate and dipropylene glycol dibenzoate and stearyl ether benzoate; glycereth-7 benzoate; octyldodecyl neopentanoate; PEG-4 diheptanoate; C12-15 alkyl lactate; benzyl laurate; octyl isononanoate; ethylhexyl isononanoate; bis trioctyldodecyl citryl; caprylic/capric triglyceride; glycereth-7 triacetate; triisostearin; isostearyl palmitate; isocetyl stearate; isononyl isononanoate; neopentyl glycol dicaprylate/dicaprate; octyldodecyl ricinoleate; ethylhexyl palmitate; octyl stearate; trimethylpropane triethylhexanoate; dioctyl sebacate; isopropyl palmitate; isopropyl myristate; isodecyl neopentanoate; isodecyl oelate; neopentyl glycol distearate; 2-ethylhexyl salicyclate; caprylic/capric triglyceride; peg-1 glycerol sorbitan isostearate; dioctyl malate; dioctyl/capryl adipate; diisopropyl sebacate; dioleyl malate; diisostearyl fumarate; diisopropyl dimer dilinoleate; dimer dilinoleyl dimer dilinoleate; dipentaerythrityl tetrahydroxystearate/isostearate; dipentaerythrityl tetrabehenate polyhydroxystearate; triisostearyl citrate; triisostearyl trilinoleate; dioctadecanyl didecyletetradecanoate; dioctadecanyl ditetradecyloctadecanoate; neopentyl glycol diethylhexanoate; neopentyl glycol dicaprate; triisostearyl citrate; stearyl erucate; behenyl erucate; isosstearyl hydroxystearate; ethylhexyl hydroxystearate; cetearyl ethylhexanoate; isostearyl isostearate; PEG-90 diisostearate; PEG/PPG-8/3 diisostearate; PEG/PPG-8/3 laurate; glycerol behenate; glycerol eicosanate; glycerol behenate eicosanate; glyceryl behenate/eicosadioate; glycerol isostearate; glyceryl tri(2-ethylhexanoate); polyglyceryl-10 behenate/eicosadioate; polyglyceryl-6 behenate; polyglyceryl-6 octastearate; polyglyceryl-6 polyricinoleate; polygyceryl-3 laurate; diisostearoyl polyglyceryl-3 dimer dilinoleate; triisostearoyl polyglyceryl-3 dimer dilinoleate; octyldodecyl ricinoleate; trioctyldodecyl citrate; castor oil; jojoba oil; corn oil; avocado oil; sunflower oil; soybean oil; olive oil; cottonseed oil; grape seed oil; palm oil; sesame seed oil; almond oil; methyl soyate; isocetyl alcohol; dipropylene glycol monomethyl ether; dipropylene glycol dimethyl ether; and dipropylene glycol methyl acetate. The product may be any one or more selected from the group consisting of lip gloss, lip stick, lip moisturizer, stick deodorant, antiperspirant, stick sun block, stick tanning lotion, stick applied pharmaceuticals, stick soaps, stick perfumes, stick applied insect repellents. In one non-limiting embodiment, the emollient comprises at least glyceryl behenate/eicosadioate or polyglyceryl-10 behenate/eicosadioate. In another non-limiting embodiment, the emollient comprises at least glyceryl behenate/eicosadioate or polyglyceryl-10 behenate/eicosadioate, and the product is selected from the group consisting of lip gloss, lip stick, and lip moisturizer.

According to even another embodiment of the present invention, there are provided methods of making and using cosmetic formulations and products.

### DETAILED DESCRIPTION OF THE INVENTION

The compositions of the present invention may include a polyamide resin and an emollient.

Polyamides suitable for use herein include any polyamides known in the art. Examples of suitable polyamides include Ester-Terminated PolyAmides (ETPAs), Tertiary-Amide-Terminated PolyAmides (ATPAs), Ester-Terminated PolyEster-Amides (ETPEAs), Tertiary Amide-Terminated PolyEster-Amides (ATPEA), PolyAlkyleneOxy-terminated PolyAmides (PAOPAs), and PolyEther-PolyAmides (PEPAs). In one embodiment, preferred polyamides are ETPAs, ATPAs, ETPEAs, and PAOPAs. A discussion of all of these polyamides may be found in U.S. Patent Publication No. 20060229222, published Oct. 12, 2006, from U.S. Pat. No. 393,387, filed Mar. 29, 2006, which is hereby incorporated by reference in its entirety.

Other non-limiting examples of ETPAs suitable for use in the present invention may be found in U.S. Pat. No. 6,864,349 for aqueous suspensions containing polymerized fatty acid-based polyamides; U.S. Pat. No. 6,517,343 for coated candles and coating compositions; and U.S. Pat. No. 6,503,077 for gelled articles containing tertiary amide-terminated polyamide, all of which are hereby incorporated by reference in their entirety.

Non-limiting examples of commercially available ETPAs suitable for use in the present invention include UNICLEAR 100 and 100VG, both available from Arizona Chemical Company.

More examples of non-limiting ATPAs suitable for use in the present invention may be found in U.S. Pat. No. 6,864,349 for aqueous suspensions containing polymerized fatty acid-based polyamides; U.S. Pat. No. 6,592,857 for tertiary amide terminated polyamides in cosmetics; 3 U.S. Pat. No. 6,517,343 for coated candles and coating compositions; U.S. Pat. No. 6,503,522 for tertiary amide-terminated polyamides in structured personal care compositions; U.S. Pat. No. 6,503,077 for gelled articles containing tertiary amide-terminated polyamide; U.S. Pat. No. 6,469,131 Structured composition containing tertiary amide-terminated polyamide for personal care products; U.S. Pat. No. 6,399,713 for hydrocarbon-terminated polyether-polyamide block copolymers and uses thereof; and U.S. Pat. No. 6,268,466 for tertiary amide terminated polyamides and uses thereof, all of which are hereby incorporated by reference.

Non-limiting examples of commercially available ATPAs suitable for use in the present invention include SLYVACLEAR A200V, A2635V and A2614V, each available from Arizona Chemical Company.

More non-limiting examples of ETPEAs suitable for use in the present invention may be found in U.S. Pat. No. 7,253,249 for ester-terminated poly(ester-amide) in personal care products; U.S. Pat. No. 6,875,245 for ester-terminated poly(ester-amides) in personal care products; and U.S. Pat. No. 6,552,160 for ester-terminated poly(ester-amides) useful for formulating transparent gels in low polarity fluids, all of which are hereby incorporated by reference.

A non-limiting example of a commercially available ETPEA suitable for use in the present invention include SYLVACLEAR C75V, available from Arizona Chemical Company.

Non-limiting examples of commercially available PAOPAs suitable for use in the present invention include SYLVACLEAR 1900V, PE1800V, WF1500V and PA1200V, each available from Arizona Chemical Company.

Non-limiting examples of emollients suitable for use in the present invention include: hydrocarbons with more than 8 carbons that may be linear or branched, and saturated or unsaturated; esters, such as benzoate esters, simple esters, or bulky esters; glycerol or glycerol condensate (mono-, di- or tri-); polyglycerol or polyglycerol condensate; guerbet ester; vegetable oils; and mixtures of silicone oils with any of the above emollients.

Non-limiting examples of hydrocarbons suitable for use herein include but are not limited to isooctane (permethyl 97A); polyisobutene and isododecane (permethyl 98B); isododecane (permethyl 99A); isohexadecane (permethyl 101A); isoeicosane (permethyl 102A); polyisobutene permethyl 104A, 106A); hydrogenated polyisobutene (panalane L-14E, from lipo); hydrogenated polyisobutene (Fancol POLYISO 200-CG, 250-CG, 275-CG, 300-CG, 450-CG, 800-CG and 1200-CG from the Fanning Corporation); paraffin; isoparaffins; petrolatum; limonene; and squalene.

Non-limiting examples of benzoate esters suitable for use herein include but are not limited to C12-C15 alkyl benzoate (Finsolv TN), isostearyl benzoate (Finsolv SB); octyl benzoate (Finsolv EB); dipropylene glycol benzoate (Dermol DPGB, from Alzo); C12-15 alkyl benzoate (and) dipropylene glycol dibenzoate (and) PPG-15 stearyl ether benzoate (Finsolv TPP from Finetex); and glycereth-7 benzoate (Palemol G-7B from Pheonix).

Non-limiting examples of simple esters to bulky esters suitable for use herein include but are not limited to octyldodecyl neopentanoate (Elefac 1205, from Bernel); PEG-4 diheptanoate (Liponate 2-DH, from Lipo); C12-15 alkyl lactate (Palemol 1215 L from Pheonix); benzyl laurate (Palemol 612 from Pheonix); octyl isononanoate (Palemol 89 from Pheonix); ethylhexyl isononanoate (Palemol 899 from Pheonix); bis trioctyldodecyl citryl (Palemol C-150 from Pheonix); caprylic/capric triglyceride (Palemol CCT from Pheonix); glycereth-7 triacetate (Palemol G-7A from Pheonix); triisostearin (Palemol GTIS from Pheonix); isostearyl palmitate (Palemol I-1816 from Pheonix); isocetyl stearate (Palemol ICS from Pheonix); isononyl isononanoate (Palemol IN-2 from Pheonix); neopentyl glycol dicaprylate/dicaprate (Palemol NPGDD from Pheonix); octyldodecyl ricinoleate (Palemol ODR from Pheonix); ethylhexyl palmitate (Palemol OP from Pheonix); octyl stearate (Palemol OS from Pheonix); trimethylpropane triethylhexanoate (Palemol TMPO from Pheonix); dioctyl sebacate (Trivent DOS from Trivent); isopropyl palmitate; isopropyl myristate; isodecyl neopentanoate; isodecyl oelate; neopentyl glycol distearate; 2-ethylhexyl salicyclate; caprylic/capric triglyceride; PEG-1 glycerol sorbitan Isostearate; dioctyl malate; dioctyl/capryl adipate; diisopropyl sebacate; dioleyl malate (Pelmol 2181M); diisostearyl fumarate; diisopropyl dimer dilinoleate (Pelmol D9336); dimer dilinoleyl dimer dilinoleate (Pelmol DD); dipentaerythrityl tetrahydroxystearate/isostearate (Pelmol DP-72); dipentaerythrityl tetrabehenate polyhydroxystearate (Pelmol DP-144B); triisostearyl citrate (Schercemol™ TISC from Lubrizol); triisostearyl trilinoleate (Schercemol™ TIST from Lubrizol); dioctadecanyl didecyletetradecanoate (Pelmol 362924); dioctadecanyl ditetradecyloctadecanoate (Pelmol 362936); neopentyl glycol diethylhexanoate (Schercemol™ NGDO from Lubrizol); neopentyl glycol dicaprate (Schercemol™ NGDC from Lubrizol); triisostearyl citrate (Schercemol™ TISC from Lubrizol); stearyl erucate (Schercemol™ SE from Lubrizol); behenyl erucate (Schercemol™ BE from Lubrizol); isosstearyl hydroxystearate (Schercemol™ SHS from Lubrizol); ethylhexyl hydroxystearate (Schercemol™ OHS from Lubrizol); cetearyl ethylhexanoate (Schercemol™ 1688 from Lubrizol); isostearyl isostearate (Schercemol™ 1818 from Lubrizol); PEG-90 diisostearate (Hydramol™ PGDS from Lubrizol); PEG/PPG-8/3 diisostearate (Hydramol™ PGPD from Lubrizol); and, PEG/PPG-8/3 laurate (Hydramol™ PGPL from Lubrizol).

Esters useful in the present invention include glycerol or glycerol condensates, which are substituted by at least one hydrocarbon (i.e., mono-, di- or tri-substitution), non-limiting examples of which include fatty acids. The hydrocarbon may be saturated or unsaturated, may be branched or linear, and/or may be substituted or unsubstituted. The glycerol or glycerol condensate may be substituted with the same or different hydrocarbon. Preferably, such an ester will have at least 4, 6, 8, 10, 12, 14, 16, 18, 20, 22, 24, 26, 28, or 30 carbon atoms, inclusive of all ranges and subranges there between. Non-limiting examples of suitable glycerol or glycerol condensates (mono-, di- or tri-) include but are not limited to: glycerol behenate (Compritol 888, Gattefosse); glycerol eicosanate; glycerol behenate eicosanate; glyceryl behenate/eicosadioate (Nomcort HK-G from The Nisshin OilliO Group, Ltd.); glycerol isostearate (Schercemol™ GMIS from Lubrizol); and, glyceryl tri(2-ethylhexanoate) (Schercemol™ GTO from Lubrizol). Certainly, glycerol and glycerol condensate may be substituted with 1, 2 or 3 substituents selected from the group of behenate, eicosanate, and diacids of eicosanate.

Non-limiting examples of polyglycerol or polyglycerol condensates, non-limiting examples of which include those derived from fatty acids, suitable for use herein include but are not limited to polyglyceryl-10 behenate/eicosadioate (Nomcort HK-P from The Nisshin OilliO Group, Ltd.), polyglyceryl-6 behenate (Pelmol 6G22); polyglyceryl-6 octastearate (Pelmol 6G818); polyglyceryl-6 polyricinoleate (Pelmol 6GPR); polygyceryl-3 laurate (Hydramol™ TGL from Lubrizol); diisostearoyl polyglyceryl-3 dimer dilinoleate (Schercemol™ PDD from Lubrizol); and, triisostearoyl polyglyceryl-3 dimer dilinoleate (Schercemol™ PTID from Lubrizol). As with the glycerol and glycerol condensates, these polyglycerols and polyglycerol condensates, may be substituted with at least one hydrocarbon. Again, the hydrocarbon may be saturated or unsaturated, may be branched or linear, and/or may be substituted or unsubstituted. The glycerol or glycerol condensate may be substituted with the same or different hydrocarbon. Preferably, such an ester will have at least 4, 6, 8, 10, 12, 14, 16, 18, 20, 22, 24, 26, 28, or 30 carbon atoms, inclusive of all ranges and subranges there between.

Non-limiting examples of guerbet esters suitable for use herein include but are not limited to octyldodecyl ricinoleate (Ultracas® G-20 from Lubrizol); and trioctyldodecyl citrate (G-66 guerbet ester from Lubrizol).

Non-limiting examples of vegetable oils suitable for use herein include but are not limited to castor oil; jojoba oil; corn oil; avocado oil; sunflower oil; soybean oil; olive oil; cottonseed oil; grape seed oil; palm oil; sesame seed oil; almond oil; and methyl soyate.

Non-limiting examples of mixtures of silicone oils with any of the above emollients suitable for use herein include but are not limited to mixture of Finsolv TN (C12-C15 alkyl benzoate) with cyclomethicone; mixture of isododecane (and) acrylates/dimethicone copolymer (KP-550 from Shin Etsu Silicones of America); and mixture of cylcomethicone, dimethicone, polyisobutene (Fancorsil® P from the Fanning Corporation).

Non-limiting examples of other suitable emollients include but are not limited to isocetyl alcohol (Dermocol I-16, from Alzo); dipropylene glycol monomethyl ether; dipropylene glycol dimethyl ether; and dipropylene glycol methyl acetate.

Non-limiting examples of emollients useful in the compositions of the present invention include (the trade name of a suitable commercial example follows in parenthesis, and it should be understood, that the present invention is not limited to the particular tradename listed): isocetyl alcohol (Dermocol I-16, from Alzo); dipropylene glycol benzoate (Dermol DPGB, from Alzo); octyldodecyl neopentanoate (Elefac 1205, from Bernel): C12-15 alkyl benzoate (and) dipropylene glycol dibenzoate (and) PPG-15 stearyl ether benzoate (Finsolv TPP from Finetex); PEG-4 diheptanoate (Liponate 2-DH, from Lipo); hydrogenated polyisobutene (Panalane L-14E, from Lipo); C12-15 alkyl lactate (Palemol 1215 L from Pheonix); benzyl laurate (Palemol 612 from Pheonix); octyl isononanoate (Palemol 89 from Pheonix); ethylhexyl isononanoate (Palemol 899 from Pheonix); bis trioctyldodecyl citryl (Palemol C-150 from Pheonix); caprylic/capric triglyceride (Palemol CCT from Pheonix); glycereth-7 triacetate (Palemol G-7A from Pheonix); glycereth-7 benzoate (Palemol G-7B from Pheonix); triisostearin (Palemol GTIS from Pheonix); isostearyl palmitate (Palemol I-1816 from Pheonix); isocetyl stearate (Palemol ICS from Pheonix); isononyl isononanoate (Palemol IN-2 from Pheonix); neopentyl glycol dicaprylate/dicaprate (Palemol NPGDD from Pheonix); octyldodecyl ricinoleate (Palemol ODR from Pheonix); ethylhexyl palmitate (Palemol OP from Pheonix); octyl stearate (Palemol OS from Pheonix); trimethylpropane triethylhexanoate (Palemol TMPO from Pheonix); dioctyl sebacate (Trivent DOS from Trivent); C12-C15 alkyl benzoate (Finsolv TN), isostearyl benzoate (Finsolv SB); octyl benzoate (Finsolv EB); mixture of Finsolv TN (C12-C15 alkyl benzoate) with cyclomethicone; isoparaffins; petrolatum; castor oil; jojoba oil; corn oil; avocado oil; sunflower oil; soybean oil; olive oil; cottonseed oil; grape seed oil; palm oil; sesame seed oil; almond oil; methyl soyate; isopropyl palmitate; isopropyl myristate; isodecyl neopentanoate; isodecyl oelate; neopentyl glycol distearate; dipropylene glycol monomethyl ether; dipropylene glycol dimethyl ether; dipropylene glycol methyl acetate; 2-ethylhexyl salicyclate; isooctane (Permethyl 97A); polyisobutene and isododecane (Permethyl 98B); isododecane (Permethyl 99A); isohexadecane (Permethyl 101A); isoeicosane (Permethyl 102A); polyisobutene (Permethyl 104A, 106A); caprylic/capric triglyceride; PEG-1 glycerol sorbitan isostearate; dioctyl malate; dioctyl/capryl adipate; diisopropyl sebacate; isododecane (and) acrylates/dimethicone copolymer (KP-550 from Shin Etsu Silicones of America).

In one non-limiting embodiment of the present invention, preferred emollients include: isocetyl alcohol; dipropylene glycol benzoate; C12-15 alkyl benzoate (and) dipropylene glycol dibenzoate (and) PPG-15 stearyl ether benzoate; PEG-4 diheptanoate; hydrogenated polyisobutene; C12-15 alkyl lactate; benzyl laurate; octyl isononanoate; ethylhexyl isononanoate; bis trioctyldodecyl citryl; caprylic/capric triglyceride; glycereth-7 triacetate; glycereth-7 benzoate; isostearyl palmitate; isocetyl stearate; octyldodecyl ricinoleate; ethylhexyl palmitate; octyl stearate; and dioctyl sebacate. C12-C15 alkyl benzoate (Finsolv TN), isostearyl benzoate (Finsolv SB); octyl benzoate (Finsolv EB); mixture of Finsolv TN (C12-C15 alkyl benzoate) with cyclomethicone; isoparaffins; petrolatum; castor oil; jojoba oil; corn oil; avocado oil; sunflower oil; soybean oil; olive oil; cottonseed oil; grape seed oil; palm oil; sesame seed oil; almond oil; methyl soyate; isopropyl palmitate; isopropyl myristate; isodecyl neopentanoate; isodecyl oelate; neopentyl glycol distearate; dipropylene glycol monomethyl ether; dipropylene glycol dimethyl ether; dipropylene glycol methyl acetate; 2-ethylhexyl salicyclate; isooctane (Permethyl 97A); polyisobutene and isododecane (Permethyl 98B); isododecane (Permethyl 99A); isohexadecane (Permethyl 101A); isoeicosane (Permethyl 102A); Polyisobutene (Permethyl 104A, 106A); caprylic/capric triglyceride; PEG-1 glycerol sorbitan isostearate; dioctyl malate; dioctyl/capryl adipate; diisopropyl sebacate; isododecane (and) acrylates/dimethicone copolymer (KP-550 from Shin Etsu Silicones of America).

In another non-limiting embodiment of the present invention, preferred emollients include: isocetyl alcohol; dipropylene glycol benzoate; C12-15 alkyl benzoate (and) dipropylene glycol dibenzoate (and) PPG-15 stearyl ether benzoate; PEG-4 diheptanoate; hydrogenated polyisobutene; C12-15 alkyl lactate; benzyl laurate; octyl isononanoate; ethylhexyl isononanoate; bis trioctyldodecyl citryl; caprylic/capric triglyceride; glycereth-7 triacetate; glycereth-7 benzoate; isostearyl palmitate; isocetyl stearate; octyldodecyl ricinoleate; ethylhexyl palmitate; octyl stearate; and dioctyl sebacate.

With respect to the weight ratios of polyamide to emollient in the compositions of the present invention, the weight ratios may range from about 0.1-90:99.9-10, preferably from about 1-80:99-2, more preferably from about 3-40:97-60. In one non-limiting embodiment, the weight ratio of polyamide to emollient in the compositions of the present invention is from about 5-30:95-70. In another non-limiting embodiment, the weight ratio of polyamide to emollient in the compositions of the present invention is from about 5-20:95-80.

The compositions of the present invention may optionally comprise any one or more ingredient commonly used in cosmetics such as a dextrin ester of an aliphatic acid. For example, mention may be made of dextrin palmitate, dextrin palmitate/octanoate. Non-limiting examples of commercial dextrin ester products suitable for use herein include Rheopearl KL and Rheopearl TT (trademarks of Chiba Seifun).

The compositions of the present invention may comprise at least one hydroxy compound selected from the group consisting of diisostearyl malate, polyglyceryl-2 diisostearate, polyglyceryl-2 triisostearate, dipentaerythrityl diisostearate, and dipentaerythrityl trisostearate. Each of these materials are commonly used in cosmetics.

The compositions of the present invention may even also optionally include conventional additives that are commonly used in personal care products. The one or more additives is utilized in such a range of amount that the effects of the present invention are not impaired. Examples of the additives suitable for use herein include pigments such as, for instance, coloring pigments such as Red No. 201 and Red No. 202, white pigments such as titanium oxide, and extender pigments such as silica; dyestuffs such as Blue No. 1, Red No. 218, and Red No. 225; lame agents; pearlescent agents; anti-oxidant agents; anti-septic agents; ultraviolet rays absorbers; perfumes; cooling agents; anti-inflammatory agents; purified water; plant oils; mineral oils; solid waxes; and silicone oils.

In some embodiments, the compositions of the present invention are useful in the making of personal care products, non-limiting example of which includes stick applied products, non-limiting examples of which include lip gloss, lip stick, lip moisturizer, stick deodorant and/or antiperspirant, stick sun block and/or tans lotions, stick applied pharmaceuticals, stick soaps, stick perfumes, stick applied insect repellents, and the like. Some embodiments of the compositions of the present invention are useful for making the carrier or base of a personal care product. Some embodiments of the present invention are useful for making a precursor of a personal care product carrier or base.

As a non-limiting example, a lip gloss may be prepared as follows. The polyamide and at least one emollient may be mixed together and dissolved under stirring, preferably, at a temperature of from 90° C. to 100° C. to obtain a homogenous mixture, which may then be subjected to defoaming. The lip gloss composition may be prepared by cooling the mixture to ambient temperature. When a colored lip gloss composition is desired, a pigment may be kneaded with an oil separately and added to the aforementioned homogeneously dissolved mixture, preferably, at a temperature of from 90° C. to 100° C. Then the mixture may be dissolved under stirring at the same temperature to obtain a homogeneous mixture, which may be then subjected to defoaming. The lip gloss composition may be prepared by cooling the mixture to ambient temperature.

### EXAMPLES

Shown in Table 1 are various non-limiting formulations of the present invention. The formulations comprise a gel comprising an ATPA (Sylvaclear A2614V from Arizona Chemical), and various emollients as listed at 20 wt % load of polyamide resin.

TABLE 1
Gel Testing of Sylvaclear A2614V in various emollients at a 20% loading
of polyamide resin.

| Chemical Name | Trade Name | Clarity | Texture |
|---|---|---|---|
| Isocetyl alcohol | Dermocol I-16 | Clear | Soluble |
| Dipropylene glycol benzoate | Dermol DPGB | Hazy | Soft |
| Octyldodecyl neopentanoate | Elefac I205 | Clear | Firm |
| C12-C15 Alkyl benozoate | Finsolv TN | Clear | Firm |
| C12-C15 Alkyl benozoate | Finsolv TPP | Clear | Medium |
| (and) dipropylene glycol | | | |
| dibenzoate (and) PPG-15 | | | |
| stearyl ether benzoate | | | |
| PEG-4 diheptanoate | Liponate 2-DH | Opaque | Soft |
| Hydrogenated Polyisobutene | Panalane L-14E | Translucent | Firm |
| C12-C15 alkyl lactate | Pelemol 1215L | Clear | Soluble |
| Benzyl laurate | Pelemol 612 | Clear | Medium |
| Octyl isononanoate | Pelemol 89 | Clear | Medium |
| 2-ethylhexyl isononanoate | Pelemol 899 | Clear | Firm |
| Bis(trioctyldodecyl citryl) | Pelemol C-150 | Clear | Medium |
| Caprylic/capric triglyceride | Pelemol CCT | Cloudy | Firm |
| Glycereth-7-triacetate | Pelemol G-7A | Incompatible | |
| Glycereth-7-benzoate | Pelemol G-7B | Incompatible | |
| Triisostearin | Pelemol GTIS | Cloudy | Medium |
| Isostearyl palmitate | Pelemol I-1816 | Hazy | Medium |
| Isocetyl stearate | Pelemol ICS | Hazy | Medium |
| Isononyl isononanoate | Pelemol IN-2 | Clear | Medium |
| Neopentyl glycol | Pelemol NPGDD | Clear | Medium |
| dicaprylate/dicaprate | | | |
| Octyldodecyl ricinoleate | Pelemol ODR | Clear | Medium |
| Ethylhexyl palmitate | Pelemol OP | Clear | Medium |
| Octyl stearate | Pelemol OS | Clear | Medium |
| Trimethylpropane | Pelemol TMPO | Clear | Firm |
| triethylhexanoate | | | |
| Dioctyl sebacate | Trivent DOS | Clear | medium |

For the various formulations shown in Table 1, desirability with respect to clarity and texture is as follows. Clarity from most desirable to least desirable is Clear>Translucent>Hazy>Cloudy>Opaque>incompatible. With respect to the property of Texture, Texture from most desirable to least desirable is Firm>Medium>Soft>Soluble. So, for applications in which Clarity is most important, most preferred is Clear/Firm, then Clear/Medium, then Clear/Soft, then Clear/Soluble, then Translucent/Firm, then Translucent/Medium, then Translucent/Soft, then Translucent/Soluble, etc. For applications in which Texture is most important, most preferred is Firm/Clear, then Firm /Translucent/then Firm/Hazy, and then of course, starting with “medium” and going through various clarity grades. It should be understood that certain combinations may work for different end use applications.

The present disclosure is to be taken as illustrative rather than as limiting the scope or nature of the claims below. Numerous modifications and variations will become apparent to those skilled in the art after studying the disclosure, including use of equivalent functional and/or structural substitutes for elements described herein, use of equivalent functional couplings for couplings described herein, and/or use of equivalent functional actions for actions described herein. Any insubstantial variations are to be considered within the scope of the claims below.

1. A cosmetic formulation comprising a polyamide and an emollient, wherein the emollient comprises at least one selected from the group consisting of hydrocarbons with more than 8 carbons that may be linear or branched, and saturated or unsaturated; esters, such as benzoate esters, simple esters, or bulky esters; glycerol or glycerol condensate (mono-, di- or tri-); polyglycerol or polyglycerol condensate; guerbet ester; vegetable oils; and mixtures of silicone oils with any of the above emollients.

2. The formulation of claim 1, wherein the emollient comprises at least one selected from the group consisting of isooctane; polyisobutene and isododecane; isododecane; isohexadecane; isoeicosane; polyisobutene; hydrogenated polyisobutene; hydrogenated polyisobutene; paraffin; isoparaffins; petrolatum; limonene; squalene; C12-C15 alkyl benzoate; isostearyl benzoate; octyl benzoate; dipropylene glycol benzoate; C12-15 alkyl benzoate and dipropylene glycol dibenzoate and stearyl ether benzoate; glycereth-7 benzoate; octyldodecyl neopentanoate; PEG-4 diheptanoate; C12-15 alkyl lactate; benzyl laurate; octyl isononanoate; ethylhexyl isononanoate; his trioctyldodecyl citryl; caprylic/capric triglyceride; glycereth-7 triacetate; triisostearin; isostearyl palmitate; isocetyl stearate; isononyl isononanoate; neopentyl glycol dicaprylate/dicaprate; octyldodecyl ricinoleate; ethylhexyl palmitate; octyl stearate; trimethylpropane triethylhexanoate; dioctyl sebacate; isopropyl palmitate; isopropyl myristate; isodecyl neopentanoate; isodecyl oelate; neopentyl glycol distearate; 2-ethylhexyl salicyclate; caprylic/capric triglyceride; PEG-1 glycerol sorbitan isostearate; dioctyl malate; dioctyl/capryl adipate; diisopropyl sebacate; dioleyl malate; diisostearyl fumarate; diisopropyl dimer dilinoleate; dimer dilinoleyl dimer dilinoleate; dipentaerythrityl tetrahydroxystearate/isostearate; dipentaerythrityl tetrabehenate polyhydroxystearate; triisostearyl citrate; triisostearyl trilinoleate; dioctadecanyl didecyletetradecanoate; dioctadecanyl ditetradecyloctadecanoate; neopentyl glycol diethylhexanoate; neopentyl glycol dicaprate; triisostearyl citrate; stearyl erucate; behenyl erucate; isosstearyl hydroxystearate; ethylhexyl hydroxystearate; cetearyl ethylhexanoate; isostearyl isostearate; PEG-90 diisostearate; PEG/PPG-8/3 diisostearate; PEG/PPG-8/3 laurate; glycerol behenate; glycerol eicosanate; glycerol behenate eicosanate; glyceryl behenate/eicosadioate; glycerol isostearate; glyceryl tri(2-ethylhexanoate); polyglyceryl-10 behenate/eicosadioate; polyglyceryl-6 behenate; polyglyceryl-6 octastearate; polyglyceryl-6 polyricinoleate; polygyceryl-3 laurate; diisostearoyl polyglyceryl-3 dimer dilinoleate; triisostearoyl polyglyceryl-3 dimer dilinoleate; octyldodecyl ricinoleate; trioctyldodecyl citrate; castor oil; jojoba oil; corn oil; avocado oil; sunflower oil; soybean oil; olive oil; cottonseed oil; grape seed oil; palm oil; sesame seed oil; almond oil; methyl soyate; isocetyl alcohol; dipropylene glycol monomethyl ether; dipropylene glycol dimethyl ether; and dipropylene glycol methyl acetate.

3. The formulation of claim 1 further comprising a cosmetically active agent.

4. The formulation of claim 1, wherein the emollient comprises at least 2 selected from the group.

5. The formulation of claim 1, wherein the emollient comprises at least 3 selected from the group.

6. The formulation of claim 1, wherein the emollient comprises at least 4 selected from the group.

7. The formulation of claim 1, wherein the weight ratio of polyamide to emollient is in the range of about 0.1-90:99.9-10.

8. The formulation of claim 1, wherein the polyamide comprises at least one selected from the group consisting of Ester-Terminated PolyAmides (ETPAs), Tertiary-Amide-Terminated PolyAmides (ATPAs), Ester-Terminated PolyEster-Amides (ETPEAs), Tertiary Amide-Terminated PolyEster-Amides (ATPEA), PolyAlkyleneOxy-terminated PolyAmides (PAOPAs), and PolyEther-PolyAmides (PEPAs).

9. The formulation of claim 8, wherein said emollient comprises at least one selected from the group consisting of glyceryl behenate/eicosadioate and polyglyceryl-10 behenate/eicosadioate.

10. A stick applied personal care product formulation comprising a colorant, a polyamide and an emollient, wherein the emollient comprises at least one selected from the group consisting of hydrocarbons with more than 8 carbons that may be linear or branched, and saturated or unsaturated; esters, such as benzoate esters, simple esters, or bulky esters; glycerol or glycerol condensate (mono-, di- or tri-); polyglycerol or polyglycerol condensate; guerbet ester; vegetable oils; and mixtures of silicone oils with any of the above emollients.

11. The formulation of claim 10, wherein the stick applied personal care product is selected from the group consisting of lip gloss, lip stick, lip moisturizer, stick deodorant, antiperspirant, stick sun block, stick tanning lotion, stick applied pharmaceuticals, stick soaps, stick perfumes, stick applied insect repellents.

12. The formulation of claim 10, wherein the emollient comprises at least one selected from the group consisting of isooctane; polyisobutene and isododecane; isododecane; isohexadecane; isoeicosane; polyisobutene; hydrogenated polyisobutene; hydrogenated polyisobutene; paraffin; isoparaffins; petrolatum; limonene; squalene; C12-C15 alkyl benzoate; isostearyl benzoate; octyl benzoate; dipropylene glycol benzoate; C12-15 alkyl benzoate and dipropylene glycol dibenzoate and stearyl ether benzoate; glycereth-7 benzoate; octyldodecyl neopentanoate; peg-4 diheptanoate; C12-15 alkyl lactate; benzyl laurate; octyl isononanoate; ethylhexyl isononanoate; bis trioctyldodecyl citryl; caprylic/capric triglyceride; glycereth-7 triacetate; triisostearin; isostearyl palmitate; isocetyl stearate; isononyl isononanoate; neopentyl glycol dicaprylate/dicaprate; octyldodecyl ricinoleate; ethylhexyl palmitate; octyl stearate; trimethylpropane triethylhexanoate; dioctyl sebacate; isopropyl palmitate; isopropyl myristate; isodecyl neopentanoate; isodecyl oelate; neopentyl glycol distearate; 2-ethylhexyl salicyclate; caprylic/capric triglyceride; PEG-1 glycerol sorbitan isostearate; dioctyl malate; dioctyl/capryl adipate; diisopropyl sebacate; dioleyl malate; diisostearyl fumarate; diisopropyl dimer dilinoleate; dimer dilinoleyl dimer dilinoleate; dipentaerythrityl tetrahydroxystearate/isostearate; dipentaerythrityl tetrabehenate polyhydroxystearate; triisostearyl citrate; triisostearyl trilinoleate; dioctadecanyl didecyletetradecanoate; dioctadecanyl ditetradecyloctadecanoate; neopentyl glycol diethylhexanoate; neopentyl glycol dicaprate; triisostearyl citrate; stearyl erucate; behenyl erucate; isosstearyl hydroxystearate; ethylhexyl hydroxystearate; cetearyl ethylhexanoate; isostearyl isostearate; PEG-90 diisostearate; PEG/PPG-8/3 diisostearate; PEG/PPG-8/3 laurate; glycerol behenate; glycerol eicosanate; glycerol behenate eicosanate; glyceryl behenate/eicosadioate; glycerol isostearate; glyceryl tri(2-ethylhexanoate); polyglyceryl-10 behenate/eicosadioate; polyglyceryl-6 behenate; polyglyceryl-6 octastearate; polyglyceryl-6 polyricinoleate; polygyceryl-3 laurate; diisostearoyl polyglyceryl-3 dimer dilinoleate; triisostearoyl polyglyceryl-3 dimer dilinoleate; octyldodecyl ricinoleate; trioctyldodecyl citrate; castor oil; jojoba oil; corn oil; avocado oil; sunflower oil; soybean oil; olive oil; cottonseed oil; grape seed oil; palm oil; sesame seed oil; almond oil; methyl soyate; isocetyl alcohol; dipropylene glycol monomethyl ether; dipropylene glycol dimethyl ether; and Dipropylene glycol methyl acetate.

13. The formulation of claim 10, wherein the emollient comprises at least 2 selected from the group.

14. The formulation of claim 10, wherein the emollient comprises at least 3 selected from the group.

15. The formulation of claim 10, wherein the emollient comprises at least 4 selected from the group.

16. The formulation of claim 10, wherein the weight ratio of polyamide to emollient is in the range of about 0.1-90:99.9-10.

17. The formulation of claim 10, wherein the polyamide comprises at least one selected from the group consisting of Ester-Terminated PolyAmides (ETPAs), Tertiary-Amide-Terminated PolyAmides (ATPAs), Ester-Terminated PolyEster-Amides (ETPEAs), Tertiary Amide-Terminated PolyEster-Amides (ATPEA), PolyAlkyleneOxy-terminated PolyAmides (PAOPAs), and PolyEther-PolyAmides (PEPAs).

18. The formulation of claim 10, wherein said emollient comprises at least one selected from the group consisting of glyceryl behenate/eicosadioate and polyglyceryl-10 behenate/eicosadioate.

19. The formulation of claim 18, wherein said stick applied personal care product is selected from the group consisting of lip gloss, lip stick, and lip moisturizer.

20. A cosmetic product comprising a cosmetically active ingredient, a polyamide and an emollient, wherein the emollient comprises at least one selected from the group consisting of hydrocarbons with more than 8 carbons that may be linear or branched, and saturated or unsaturated; esters, such as benzoate esters, simple esters, or bulky esters; glycerol or glycerol condensate (mono-, di- or tri-); polyglycerol or polyglycerol condensate; guerbet ester; vegetable oils; and mixtures of silicone oils with any of the above emollients.

21. The product of claim 20, wherein the product is selected from the group consisting of lip gloss, lip stick, lip moisturizer, stick deodorant, antiperspirant, stick sun block, stick tanning lotion, stick applied pharmaceuticals, stick soaps, stick perfumes, and stick applied insect repellents.

22. The product of claim 20, wherein the emollient comprises at least one selected from the group consisting of isooctane; polyisobutene and isododecane; isododecane; isohexadecane; isoeicosane; polyisobutene; hydrogenated polyisobutene; hydrogenated polyisobutene; paraffin; isoparaffins; petrolatum; limonene; squalene; C12-C15 alkyl benzoate; isostearyl benzoate; octyl benzoate; dipropylene glycol benzoate; C12-15 alkyl benzoate and dipropylene glycol dibenzoate and stearyl ether benzoate; glycereth-7 benzoate; octyldodecyl neopentanoate; PEG-4 diheptanoate; C12-15 alkyl lactate; benzyl laurate; octyl isononanoate; ethylhexyl isononanoate; bis trioctyldodecyl citryl; caprylic/capric triglyceride; glycereth-7 triacetate; triisostearin; isostearyl palmitate; isocetyl stearate; isononyl isononanoate; neopentyl glycol dicaprylate/dicaprate; octyldodecyl ricinoleate; ethylhexyl palmitate; octyl stearate; trimethylpropane triethylhexanoate; dioctyl sebacate; isopropyl palmitate; isopropyl myristate; isodecyl neopentanoate; isodecyl oelate; neopentyl glycol distearate; 2-ethylhexyl salicyclate; caprylic/capric triglyceride; PEG-1 glycerol sorbitan isostearate; dioctyl malate; dioctyl/capryl adipate; diisopropyl sebacate; dioleyl malate; diisostearyl fumarate; diisopropyl dimer dilinoleate; dimer dilinoleyl dimer dilinoleate; dipentaerythrityl tetrahydroxystearate/isostearate; dipentaerythrityl tetrabehenate polyhydroxystearate; triisostearyl citrate; triisostearyl trilinoleate; dioctadecanyl didecyletetradecanoate; dioctadecanyl ditetradecyloctadecanoate; neopentyl glycol diethylhexanoate; neopentyl glycol dicaprate; triisostearyl citrate; stearyl erucate; behenyl erucate; isosstearyl hydroxystearate; ethylhexyl hydroxystearate; cetearyl ethylhexanoate; isostearyl isostearate; PEG-90 diisostearate; PEG/PPG-8/3 diisostearate; PEG/PPG-8/3 laurate; glycerol behenate; glycerol eicosanate; glycerol behenate eicosanate; glyceryl behenate/eicosadioate; glycerol isostearate; glyceryl tri(2-ethylhexanoate); polyglyceryl-10 behenate/eicosadioate; polyglyceryl-6 behenate; polyglyceryl-6 octastearate; polyglyceryl-6 polyricinoleate; polygyceryl-3 laurate; diisostearoyl polyglyceryl-3 dimer dilinoleate; triisostearoyl polyglyceryl-3 dimer dilinoleate; octyldodecyl ricinoleate; trioctyldodecyl citrate; castor oil; jojoba oil; corn oil; avocado oil; sunflower oil; soybean oil; olive oil; cottonseed oil; grape seed oil; palm oil; sesame seed oil; almond oil; methyl soyate; isocetyl alcohol; dipropylene glycol monomethyl ether; dipropylene glycol dimethyl ether; and dipropylene glycol methyl acetate.

23. The product of claim 20, wherein the emollient comprises at least 2 selected from the group.

24. The product of claim 20, wherein the emollient comprises at least 3 selected from the group.

25. The product of claim 20, wherein the emollient comprises at least 4 selected from the group.

26. The product of claim 20, wherein the weight ratio of polyamide to emollient is in the range of about 0.1-90:99.9-10.

27. The product of claim 20, wherein the polyamide comprises at least one selected from the group consisting of Ester-Terminated PolyAmides (ETPAs), Tertiary-Amide-Terminated PolyAmides (ATPAs), Ester-Terminated PolyEster-Amides (ETPEAs), Tertiary Amide-Terminated PolyEster-Amides (ATPEA), PolyAlkyleneOxy-terminated PolyAmides (PAOPAs), and PolyEther-PolyAmides (PEPAs).

28. The product of claim 27, wherein said emollient comprises at least one selected from the group consisting of glyceryl behenate/eicosadioate and polyglyceryl-10 behenate/eicosadioate.

29. The product of claim 28, wherein said product is selected from the group consisting of lip gloss, lip stick, and lip moisturizer.
